(19) United States
(12) Patent Application Publication
(10) Pub. No.: US 2025/0255499 A1
(43) Pub. Date: Aug. 14, 2025

(54) BLOOD PRESSURE MEASUREMENT

(71) Applicant: Nokia Technologies Oy, Espoo (FI)

(72) Inventors: Bibek SAMANTA, Murray Hill, NJ (US); Hongwei LI, Cambridge (GB)

(21) Appl. No.: 19/022,138

(22) Filed: Jan. 15, 2025

(30) Foreign Application Priority Data
Feb. 9, 2024 (GB) 2401803.8

(51) Int. Cl. A61B 5/021; A61B 5/00; A61B 5/026; A61B 5/0295

(52) U.S. Cl. CPC A61B 5/02141; A61B 5/0261; A61B 5/0295; A61B 5/6887; A61B 2560/0462

(57) ABSTRACT

Examples of the disclosure relate to apparatus for blood pressure measurement. The apparatus comprise interferometric means and blood flow monitoring means. The interferometric means comprises a deformable surface wherein the deformable surface is at least partially reflective and is configured to be deformed by a user's body part. The interferometric means are configured to measure deformation of the deformable surface by the user's body part. The blood flow monitoring means are configured to enable monitoring of blood flow in the user's body part. The apparatus also comprises means for providing an output of the interferometric means and an output of the blood flow monitoring means to enable calculation of the user's blood pressure using the respective outputs.

### TECHNOLOGICAL FIELD

Examples of the disclosure relate to blood pressure measurement. Some examples of the disclosure relate to apparatus that can be used to measure blood pressure by a non-trained user such as a consumer as opposed to trained health care professional such as a doctor or nurse.

### BACKGROUND

Blood pressure measurements can be used to monitor health conditions of a user. Apparatus that can be used to measure blood pressure by a non-trained user such as a consumer as opposed to a trained health care professional can enable such measurements to be made at the user's convenience.

### BRIEF SUMMARY

According to various, but not necessarily all, examples of the disclosure there is provided an apparatus comprising:
interferometric means comprising at least one deformable surface wherein the deformable surface is at least partially reflective and is configured to be deformed by a user's body part and wherein the interferometric means are configured to measure deformation of the deformable surface by the user's body part;
blood flow monitoring means configured to enable monitoring of blood flow in the user's body part; and
means for providing an output of the interferometric means and an output of the blood flow monitoring means to enable calculation of the user's blood pressure using the respective outputs.

The blood flow monitoring means may comprise one or more photoplethysmography sensors.

The one or more photoplethysmography sensors may comprise a light source configured to direct light towards the user's body part and one or more sensors configured to detect light scattered from the user's body part.

The blood flow monitoring means may be configured so that light used by the blood flow monitoring means is transmitted through at least part of the at least one deformable surface.

The apparatus may comprise one or more light sources wherein the one or more light sources are configured to provide beams of light for at least one of:
the interferometric means;
the blood flow monitoring means.

The one or more light sources may be configured to provide beams of light in a first wavelength range for the interferometric means and beams of light in a second wavelength range for the blood flow monitoring means.

The interferometric means may comprise a sensor configured to detect light reflected from the deformable surface and light reflected from a fixed reflective surface.

The apparatus may comprise means for providing instructions to the user to indicate a force to be applied to the deformable surface by the user's body part.

The user's body part may comprise a digit.

The apparatus may be at least one of:
comprised within a user electronic device;
an attachment for a user electronic device;
an independent device.

According to various, but not necessarily all, examples of the disclosure there is provided an apparatus comprising:
one or more interferometers comprising at least one deformable surface wherein the deformable surface is at least partially reflective and is configured to be deformed by a user's body part and wherein the one or more interferometers are configured to measure deformation of the deformable surface by the user's body part;
one or more blood flow monitors configured to enable monitoring of blood flow in the user's body part; and
wherein the apparatus is configured to provide an output of the one or more interferometers and an output of the one or more blood flow monitors to enable calculation of the user's blood pressure using the respective outputs.

According to various, but not necessarily all, examples of the disclosure there is provided an apparatus comprising means for:
obtaining an interferometric signal where the interferometric signal indicates a deformation of deformable surface where the deformation is caused by a user's body part pressing against the deformable surface;
obtaining a blood flow monitoring signal where the signal is obtained from one or more sensors configured to monitor blood flow in the user's body part; and
processing the interferometric signal and the blood flow monitoring signal to calculate the user's blood pressure.

The interferometric signal and the blood flow monitoring signal may be are obtained from measurements made within simultaneous time windows.

The blood flow monitoring signal may be obtained for a range of deformations of the deformable surface.

The range of deformations of the deformable surface may correspond to a range of pressures comprising at least one of:
diastolic pressure,
mean pressure,
systolic pressure.

The apparatus may comprise means for providing instructions to the user to indicate a force to be applied to the deformable surface by the user's body part such that the range of deformations are achieved.

According to various, but not necessarily all, examples of the disclosure there is provided a method comprising:
obtaining an interferometric signal where the interferometric signal indicates a deformation of deformable surface where the deformation is caused by a user's body part pressing against the deformable surface;
obtaining a blood flow monitoring signal where the signal is obtained from one or more sensors configured to monitor blood flow in the user's body part; and
processing the interferometric signal and the blood flow monitoring signal to calculate the user's blood pressure.

A computer program comprising instructions which, when executed by an apparatus, cause the apparatus to perform at least:
obtaining an interferometric signal where the interferometric signal indicates a deformation of deformable surface where the deformation is caused by a user's body part pressing against the deformable surface;
obtaining a blood flow monitoring signal where the signal is obtained from one or more sensors configured to monitor blood flow in the user's body part; and
processing the interferometric signal and the blood flow monitoring signal to calculate the user's blood pressure.

According to various, but not necessarily all, examples of the disclosure there is provided a system comprising:
an apparatus as described herein;
   and
at least one processor configured to process an output of the interferometric means and an output of the blood flow monitoring means to calculate the user's blood pressure.

According to various, but not necessarily all, examples of the disclosure there is provided a system comprising:
an apparatus as described herein;
   and
means for processing an output of the interferometric means and an output of the blood flow monitoring means to calculate the user's blood pressure.

### BRIEF DESCRIPTION

Some examples will now be described with reference to the accompanying drawings in which:

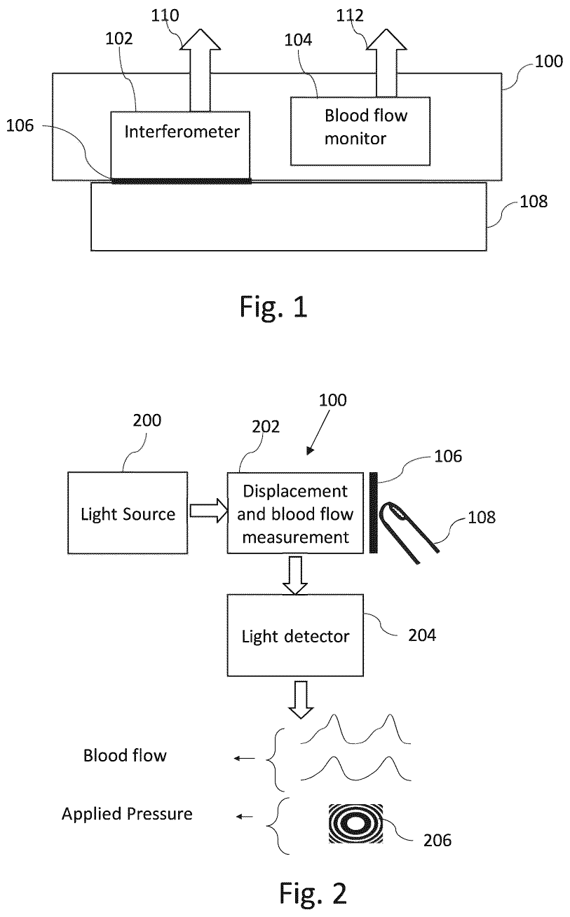

FIG. 1 shows an example apparatus:

FIG. 2 shows an example apparatus;

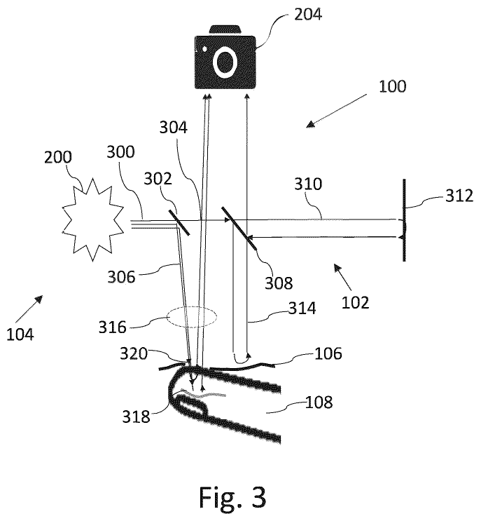

FIG. 3 shows an example apparatus;

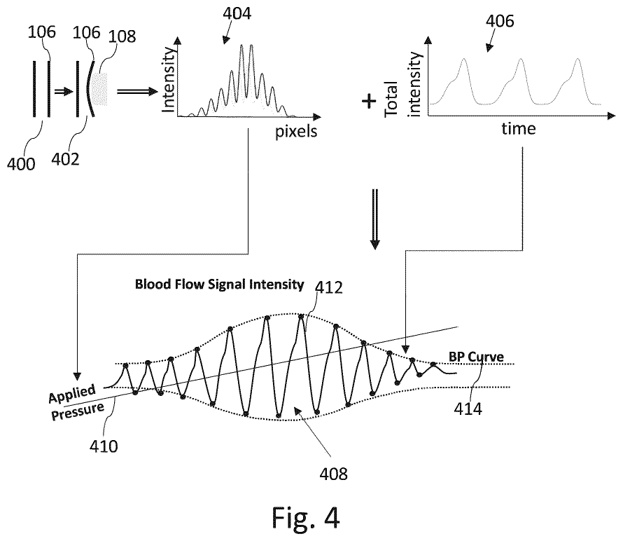

FIG. 4 shows example measurements;

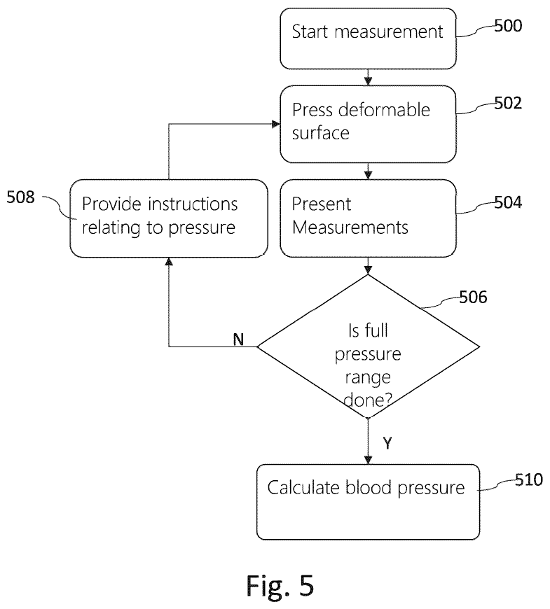

FIG. 5 shows an example method;

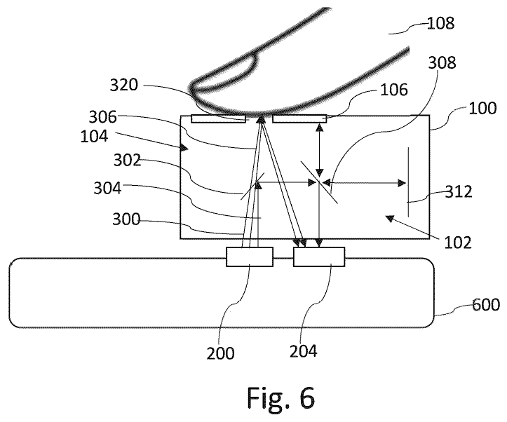

FIG. 6 shows an example apparatus;

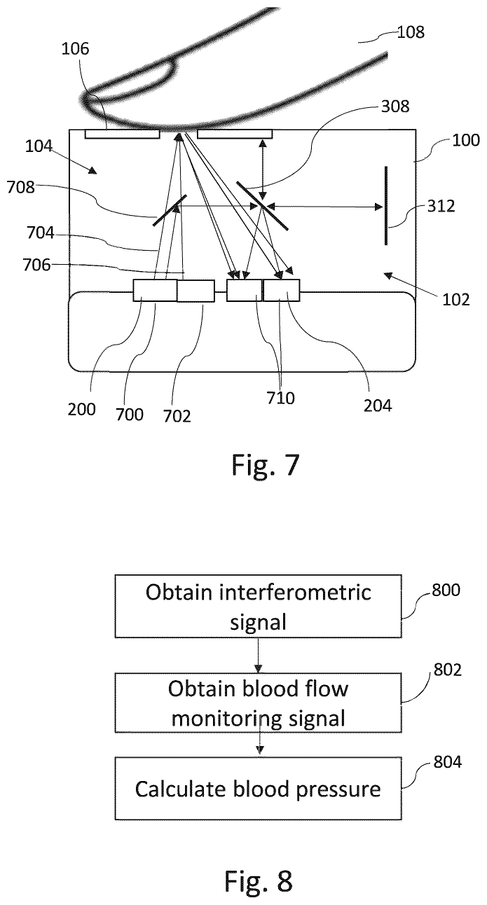

FIG. 7 shows an example apparatus;

FIG. 8 shows an example method;

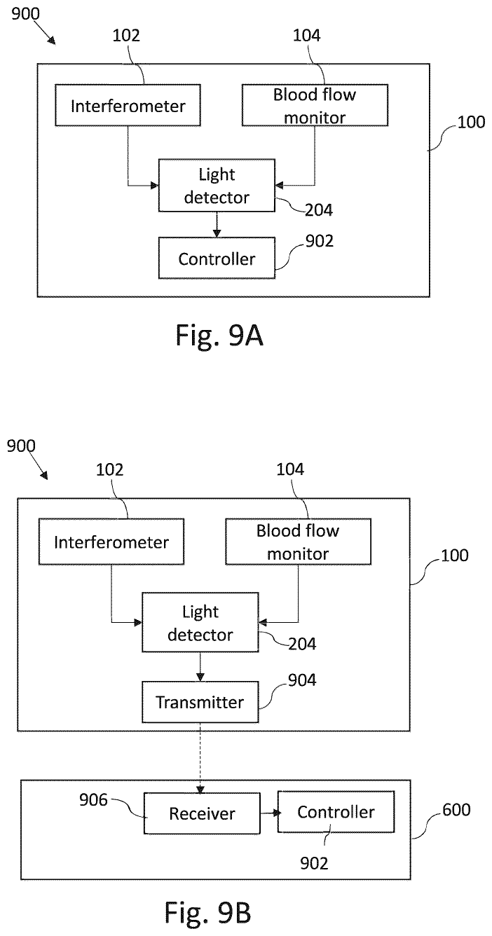

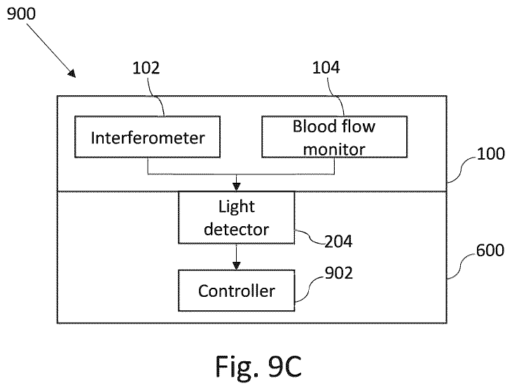

FIGS. 9A to 9C shows example systems; and

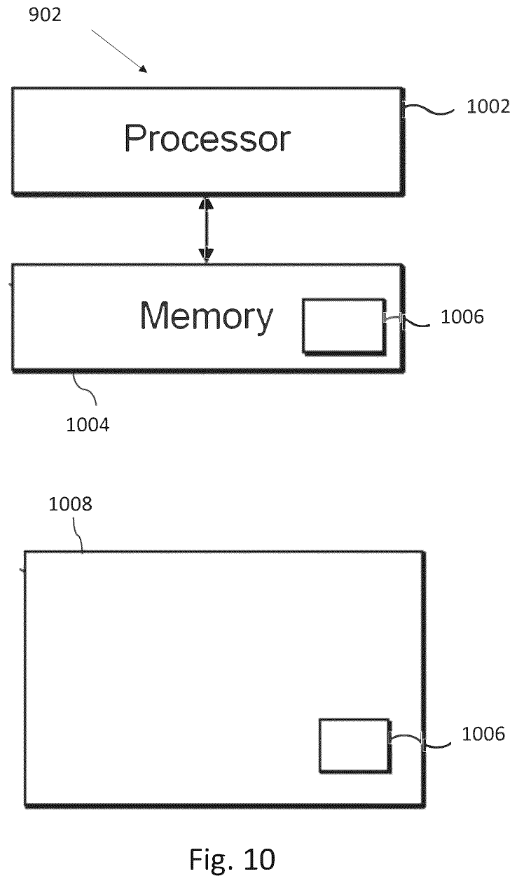

FIG. 10 shows an example controller.

The figures are not necessarily to scale. Certain features and views of the figures can be shown schematically or exaggerated in scale in the interest of clarity and conciseness. For example, the dimensions of some elements in the figures can be exaggerated relative to other elements to aid explication. Corresponding reference numerals are used in the figures to designate corresponding features. For clarity, all reference numerals are not necessarily displayed in all figures.

### DETAILED DESCRIPTION

Blood pressure measurements can be used to assist in monitoring a range of health and physical conditions. Hypertension is a leading cause of death globally due to elevated risks of cardiovascular diseases including stroke, heart disease and chronic kidney conditions. Monitoring blood pressure can help to prevent such deaths and serious illnesses.

Apparatus that can be used by a consumer to measure their own blood pressure can enable users to measure their own blood pressure without requiring assistance from a health care professional. This can enable a user to make measurements of their own blood pressure at their own convenience which can provide useful information and can enable early interventions to be provided.

Examples of the disclosure relate to such apparatus for measuring blood pressure. The apparatus is simple to use and so does not need to be operated by a health care professional. The apparatus can provide accurate and reliable results. The apparatus does not need to be calibrated by the user.

FIG. 1 schematically shows an example apparatus 100 that can be used for measuring blood pressure. Only parts of the apparatus 100 that are referred to in the following description are shown in FIG. 1. The apparatus 100 can comprise other components that are not shown in FIG. 1.

In some examples the apparatus 100 can be an independent device that comprises all components needed to make the blood pressure measurements within the apparatus 100. In such examples the apparatus 100 could comprise components such as a controller that is not shown in FIG. 1.

In some examples the apparatus 100 could be part of another device such as a user electronic device. For example, the apparatus 100 could be a module that could be comprised within the another device. In some examples the apparatus 100 could be an attachment that is configured to be attached to another device such as a user electronic device. The attachment could be removably attached by a user. In such examples the user electronic device could comprise a controller and the apparatus 100 could be configured to send signals to the controller for calculating a blood pressure measurement. In some examples some of the components that are needed to make the blood pressure measurements, such as the light source or the detectors can be part of the another device. The another device could be a user electronic device such as a smart phone or any other suitable type of device.

In the example of FIG. 1 the apparatus 100 comprises interferometric means 102 and blood flow monitoring means 104.

The interferometric means 102 can be configured to enable a force or pressure applied to the deformable surface 106 by a user's body part 108 to be measured. The force or pressure applied by the user's body part 108 on the deformable surface 106 is equal to the force or pressure experienced by the user's body part 108. The interferometric means 102 can comprise one or more interferometers and/or any other suitable means. The interferometric means can comprise a Michelson interferometer, Fizeau interferometer, or a Fabry Perot interferometer or any other suitable type of interferometer. The pressure can be measured by measuring a displacement and then using known information about the material properties of the displaced surface to calculate the force or pressure.

In some examples the interferometric means 102 can comprise one or more complete interferometers. In such cases the interferometric means 102 can comprise a light source, appropriately arranged optical components and a detector.

In some examples the interferometric means 102 can comprise part of an interferometer. For example, the interferometric means 102 could comprise the reflective surfaces or other optical components of an interferometer but the light source and/or light detector could be provided in another device to which the apparatus 100 can be connected, attached or comprised within. For example, a light source and/or detector of a camera of a smart phone could be used to provide a light source and/or detector of the interferometer.

The interferometric means 102 comprises at least one deformable surface 106. The deformable surface 106 is at least partially reflective. The deformable surface 106 can be at least partially reflective so that the deformable surface 106 provides one of the reflective surfaces of the interferometric means 102. The deformable surface 106 is at least partially reflective so that the deformation of the deformable surface 106 is detected by the interferometric means 102.

The interferometric means 102 can also comprise a sensor configured to detect light reflected from the deformable surface and light reflected from a fixed reflective surface. In other examples the detector could be provided as part of a device to which the apparatus 100 can be connected, attached or comprised within.

The deformable surface 106 is configured to be deformed by a user's body part 108. The deformable surface 106 can be positioned in the apparatus 100 so that a user can deform the deformable surface 106 by pressing their body part 108 against the deformable surface 106. In some examples the deformable surface 106 is part of an external housing of the apparatus 100 so that a user can touch the deformable surface when the apparatus 100 is in use. The user can make direct contact with the deformable surface 106.

In examples of the disclosure the user's body part 108 can be a digit such as a finger or any other suitable part of the body that can be pressed against the deformable surface 106.

The interferometric means 102 are configured to measure deformation of the deformable surface 106 by the user's body part 108. The measurement of the deformation of the deformable surface 106 by the user's body part gives an indication of the force applied by the user's body part 108.

The apparatus 100 also comprises blood flow monitoring means 104 configured to enable monitoring of blood flow in the user's body part 108. The blood flow monitoring means 104 can comprise a blood flow monitor such as one or more photoplethysmography sensors or any other suitable means. The one or more photoplethysmography sensors can comprise a light source and/or any other optical components configured to direct light towards the user's body part and one or more sensors configured to detect light scattered from the user's body part.

In some examples the blood flow monitoring means 104 can comprise one or more complete blood flow monitors. In such cases the blood flow monitoring means 104 can comprise a light source, appropriately arranged optical components and a detector.

In some examples the blood flow monitoring means 104 can comprise part of a blood flow monitor. For example, the blood flow monitoring means 104 could comprise the optical components that direct a beam of light of appropriate wavelength to the user's body part but the light source and/or light detector could be provided in another device to which the apparatus 100 can be attached or comprised within. For example, a light source and/or detector of a camera of a user device could be used as part of the blood flow monitoring means 104. In some examples the light source and/or detector could be shared by the interferometric means 102 and the blood flow monitoring means 104.

In the example of FIG. 1 the interferometric means 102 and the blood flow monitoring means 104 are shown side by side for clarity. The interferometric means 102 and the blood flow monitoring means 104 can be configured so that they can both obtain measurements from the same region of the user's body part 108. That is, the blood flow monitoring means 104 can be configured so that it measures the blood flow in the region of the user's body part 108 that is in contact with the deformable surface 106. In some examples, the blood flow monitoring means 104 can be configured so that light used by the blood flow monitoring means 104 is transmitted through at least part of the deformable surface 106. In such examples the deformable surface 106 can be at least partially transparent to the wavelengths of light that are used for blood flow monitoring. In some examples the deformable surface 106 can comprise small gaps, perforations, or discontinuities through which allow at least some of the light used for blood flow monitoring to pass through.

The apparatus 100 is also configured to provide an output 110 of the interferometric means 102 and an output 112 of the blood flow monitoring means 104. The outputs 110, 112 enable calculation of the user's blood pressure. The respective outputs can comprise any suitable signals. For instance, if the interferometric means 102 and/or the blood flow monitoring means 104 comprise a light detector then the respective outputs 110, 112 can comprise an electric signal. The electric signals can be provided to a controller or any other suitable means to be processed to determine the user's blood pressure. If the interferometric means 102 and/or the blood flow monitoring means 104 do not comprise a light detector then the respective output 110, 112 can comprise an optical signal. The optical signal can be provided to a suitable light detector to be converted to an electrical signal before it is processed by a controller to determine the user's blood pressure. The light detector could be part of the apparatus 100 or part of a user electronic device that can be connected, attached or comprised within the apparatus 100 or can be part of any other suitable means.

In some examples the apparatus 100 can comprise one or more light sources. The light sources can be part of the interferometric means 102 and/or the blood flow monitoring means 104. In some examples the one or more light sources can be shared by the interferometric means 102 and the blood flow monitoring means 104

In some examples the one or more light sources can be provided separately to the interferometric means 102 and/or the blood flow monitoring means 104. For instance, a light source could be provided in another device, such as a smart phone or camera, to which the apparatus 100 can be connected, attached or comprised within. The apparatus 100 can be configured so that the light from the external light sources can provide beams of light for the interferometric means 102 and/or the blood flow monitoring means 104.

In some examples the light sources can be arranged to provide light in different wavelengths for the different means. For instance, the light sources can be configured to provide beams of light in a first wavelength range for the interferometric means 102 and beams of light in a second wavelength range for the blood flow monitoring means 104. The first wavelength range could comprise blue light or any other suitable wavelengths of light. The second wavelength range could comprise, red light, green light, infra-red light or any other suitable wavelengths of light.

In some examples the apparatus 100 can comprise means for providing instructions to the user to indicate a force to be applied to the deformable surface 106 by the user's body part 108. The instructions can indicate to the user if they need to press harder or lighter and how long they need to maintain a given force for. In some examples the instructions can be provided by an output device. The output device could be a screen for displaying visual instructions or a loudspeaker for providing audible instructions. The output device could be part of the apparatus 100. In other examples the output device could be part of a device to which the apparatus 100 is connected, attached or comprised within. The output device could be wirelessly connected to the apparatus using any suitable wireless communications protocol.

FIG. 2 schematically shows a principle of operation of an example apparatus 100. The apparatus 100 can comprise components as shown in FIG. 1 or could have any other suitable arrangement. Corresponding reference numerals are used for corresponding features.

A light source 200 is provided. The light source 200 can be part of the apparatus 100 or can be part of another device to which the apparatus 100 can be attached or comprised within. The another device could be a user electronic device.

The light source 200 can provide light in any suitable wavelength ranges. In some examples the light source 200 can provide light in a first wavelength range for the interferometric measurements and can provide light in a second wavelength range for the blood flow measurement. The first wavelength range and the second wavelength range can be different. The first wavelength range can comprise blue light or any other suitable wavelengths. The second wavelength range can comprise red light, green light, infra-red light or any other suitable wavelengths. The wavelengths that are used for the respective ranges can depend upon the type of sensors available for detecting the light and/or any other suitable factors. In some examples the light source 200 can comprise a broadband source that can provide light across the different wavelength ranges.

The light source 200 can comprise one or more light emitting diodes or any other suitable light sources.

The apparatus 100 is configured so that light from the light source 200 is used by a measurement block 202. The measurement block 202 is configured for measuring the displacement of a deformable surface 106 and the user's blood flow. The measurement block 202 can comprise the interferometric means 102 and the blood flow monitoring means 104 which can be as shown in FIG. 1.

The deformable surface 106 can be part of the interferometric means 102. The deformable surface 106 is at least partially reflective so that the deformable surface 106 provides a reflective surface within an interferometer.

The deformable surface 106 is configured to be displaced by a user's body part pressing against the deformable surface 106. In this case the user's body part 108 comprises a finger. The user can press their fingertip against the deformable surface 106 so as to cause bending or other deformation of the deformable surface 106. The displacement caused by the user pressing their body part 108 against the deformable surface 106 can be measured using the interferometric means 102.

The blood flow monitoring means 104 can comprise one or more photoplethysmography sensors or any other suitable means. The blood flow measurement can be made by directing light from the light source 200 onto the user's body part 108 and detecting the reflected light to measure changes in blood volume within the user's body part 108.

In examples of the disclosure the same region of the user's body part 108 is used to deform the deformable surface 106 and also used for the blood flow measurement. Therefore, the light used for the blood flow measurement is incident on the same region of the user's body part 108 that is in contact with the deformable surface 106. The apparatus is configured so that light from the light source 200 is directed towards the appropriate part of the user's body. In this example the user's fingertip is used to press against the deformable surface 106 and also used for the blood flow measurement.

The light from the measurement block 202 is provided to a light detector 204. The light detector 204 can be part of the apparatus 100 or can be part of another device to which the apparatus 100 can be attached or comprised within. The light detector 204 can be part of interferometric means 102, the blood flow monitoring means 104 or can be separate from these means.

The light detector 204 can comprise any means that can be configured to transduce incident light into corresponding electrical output signals. The light detector 204 can comprise a sensor from a camera module or a user electronic device. The light detector 204 can comprise a charge coupled device and/or any other suitable means.

The outputs of the light detector 204 can be processed to provide a measurement of the pressure applied by the user's body part 108. The pressure applied by the user's body part can be determined from changes in an interference pattern 206 detected by the light detector. The applied pressure can be determined from changes in the fringes of the interference pattern 206.

The outputs of the light detector 204 can be processed to provide a measurement of the blood flow within the user's body part 108. This can be obtained from the output of the blood flow monitoring means 104.

The user's blood pressure can be determined by measuring the intensity of the blood flow signals against the applied pressure. This can enable both systolic and diastolic blood pressure to be measured.

In examples where a first wavelength of light is used for measuring the displacement of the deformable surface 106 and the second wavelength of light is used for measuring the blood flow, the same light detector 204 can be used for both measurements. In such cases the channels of the light detector 204 corresponding to the first wavelength of light (for example the blue channel) would provide the applied pressure information and the channels of the light detector 204 corresponding to the second wavelength of light (for example the red and/or green channel) would provide the blood flow information.

FIG. 3 shows an example lay out of an apparatus 100. The apparatus 100 comprises interferometric means 102 and blood flow monitoring means 104 which can be as shown in FIGS. 1 and 2.

In the example of FIG. 3 a light source 200 provides a broadband beam of light 300. The broadband beam of light 300 can comprise multiple wavelength ranges. The broadband beam of light 300 can comprise a first wavelength range that can be used for interferometric measurements and a second wavelength range that can be used for blood flow measurements. The respective wavelength ranges can comprise different wavelengths.

In the example of FIG. 3 the broadband beam of light 300 is provided to a dichroic filter 302. The dichroic filter 302 is configured to split the broadband beam of light 300 into a first wavelength beam 304 and a second wavelength beam 306. In the example of FIG. 3 the dichroic filter 302 enables the first wavelength beam 304 to pass through but does not enable the second wavelength beam 306 to pass through. Other types of filters or beam splitters could be used in other examples.

The first wavelength beam 304 is used by the interferometric means 102 to measure displacement of a deformable surface 106.

In this example the interferometric means 102 comprises a Michelson interferometer. Other types of common path interferometers, such as Fizeau interferometers or Fabry-Perot interferometers, can be used in other examples. In the example of FIG. 3 the interferometric means 102 comprises a beam splitter 308, a fixed reflective surface 312 and a deformable surface 106.

The interferometric means 102 is arranged so that the first wavelength beam 304 is incident on the beam splitter 308. The beam splitter 308 can comprise any optical components that can be configured to split the first wavelength beam 304 into a first part 310 and a second part 314. The first part 310 is directed towards the fixed reflective surface 312 and the second part 314 is directed towards the deformable surface 106.

The deformable surface is, at least partially reflective so that the second part 314 of the beam of light is at least partially reflected.

The apparatus 100 also comprises a light detector 204. The light detector 204 can be part of the interferometric means 102 or part or a user electronic device or any other suitable device. In some examples the light detector 204 can be a light detector 204 of a camera module or of any other part of a user electronic device. This enables existing components of an electronic device to be reused for the purpose of measuring blood pressure.

The interferometric means 102 is configured so that the light reflected from the deformable surface 106 and the light reflected from the fixed reflective surface 312 is incident on the light detector 204. The light detector 204 can detect an interference pattern 206 of the respective reflected parts of the beam of the light. The fringes in the interference pattern will depend upon the deformation of the deformable surface 106. Therefore, the interference pattern gives an indication of the deformation of the deformable surface 106. The force applied by the user's body part 108 to the deformable surface 106 can be determined from the deformation of the deformable surface 106 and known material properties of the deformable surface 106 such as area, thickness, Youngs modulus, bulk compressibility and/or any other relevant material properties or values based on material properties. The pressure against the user's body part 108 can then be inferred from the force applied using the known area of the deformable surface 106.

The second wavelength beam 306 is used by the blood flow monitoring means 104 to measure blood flow in the user's body part 108.

In this example the blood flow monitoring means 104 comprises a photoplethysmography sensor. Other types of blood flow monitoring means 104 can be used in other examples. In the example of FIG. 3 the blood flow monitoring means 104 comprises one or more optical components 316 configured to direct the second wavelength beam 306 towards the deformable surface 106.

The deformable surface 106 can be at least partially transparent to the second wavelength beam 306. For instance, the deformable surface 106 can be made of a material that reflects the first wavelength of light but allows the second wavelength of light to pass through. In some examples the deformable surface 106 can comprise one or more holes 320 that the second wavelength beam 306 can pass through. An example hole 320 is shown in FIG. 3. This hole 320 is not shown to scale. The hole 320 would be small in actual implementation to allow the second wavelength beam 306 to pass through to the user's body part 108, while still allowing the deformable surface 106 to be deformed by the user's body part 108.

The second wavelength beam 306 passes through the deformable surface 106 and is incident on the user's body part 108. The second wavelength beam 306 is either absorbed or reflected by the blood vessels 318 in the user's body part 108. The amount of light that is absorbed or reflected depends on the volume of blood in the blood vessels 318 in the user's body part 108. Therefore, the amount of light reflected gives an indication of blood flow in the user's body part 108.

In the example of FIG. 3 the user's body part 108 comprises a finger. Other parts of the body could be used in other examples.

The light reflected by the user's body part 108 for blood flow measurements can be detected by a light detector 204. In the example of FIG. 3 the same light detector 204 is used to detect light from the interferometric means 102 and also from the blood flow monitoring means. In this example different wavelengths of light are used for the blood flow measurement and the applied pressure. Therefore, the light detector 204 can provide a first output, corresponding to the first wavelength, that comprises information of the applied pressure and a second output, corresponding to the second wavelength, that comprises information about the blood flow.

FIG. 4 shows the relationship between the blood flow measurements, the applied pressure and the user's blood pressure.

At block 400 the deformable surface 106 of the apparatus 100 is not deformed. In this case, in the undeformed state the deformable surface 106 is flat or substantially flat. The initial interference pattern can be measured at this point while the deformable surface is not deformed.

At block 402 a user presses their body part 108 such as a finger against the deformable surface 106. The pressure applied by the body part 108 causes deformation of the deformable surface 106. In the deformed state the deformable surface 106 is at least partially curved. The extent of the curvature can range from nanometers to millimeters depending on factors such as the material properties of the deformable surface 106, the sensitivity of the interferometric means 102 and/or any other relevant factors. This deformation changes the optical path length of one of the arms of the interferometric means 102 and so changes the fringes in the detected interference pattern 206.

The fringes in the interference pattern can be detected as a variation in intensity of respective pixels of the light detector 204. An example plot 404 of the variation in intensity for respective pixels of the light detector 204 is shown in FIG. 4.

The user can change the pressure that they apply to the deformable surface 106. For instance, a user can begin by applying a light pressure but can increase this. In some examples the apparatus 100, or a device to which the apparatus 100 is connected attached or within, can provide instructions to the user instructing the user how much pressure to apply. The instructions can be provided audially, visually or by any other suitable means.

The range of applied pressures can comprise a light touch that does not deform the deformable surface 106 and does not change the blood flow in the user's body part 108 and a hard press that restricts blood flow in the user's body part so as to enable blood pressure to be measured.

The variations in pressure applied by the user's body part 108 provide variations in the deformation of the deformable surface 106. The variations in the deformation of the deformable surface 106 produce changes to the fringes in the interference pattern that can be detected. This can be detected as changes in the intensity detected by respective pixels of the light detector 204.

The user can vary the pressure applied by their body part 108 over a time window. That is, there might be a time window of several seconds over which the measurements of the applied pressure are made. During the same time window, the blood flow measurements can be made. The blood flow measurements can be made while the user is applying the pressure to the deformable surface 106. The timings of the samples used to measure the applied pressure and the blood flow do not need to be exactly the same, provided that the effects of the pressure applied to the deformable surface 106 are occurring when the blood flow measurements are made.

An example plot 406 of the blood flow measurements is shown in FIG. 4. These can be obtained in the same time window for which the deformable surface 106 is deformed. The plot 406 shows the pulse of the user. The magnitude of the peaks in the pulses can change as the user applies pressure to the deformable surface 106.

The plot 408 shows how the intensity of the peaks in the blood flow signal change as the applied pressure is varied. The trace 410 shows the applied pressure. This is shown as a straight line. When the apparatus 100 is in use the applied pressure does not need to be increased in a linear fashion but multiple measurement points can be made as the applied pressure is varied. Instructions can be provided to a user if measurements within a particular range of applied pressures need to be made or re-made.

The trace 412 shows how the blood flow signal intensity 412 changes as the applied pressure is increased.

At low applied pressure, blood flow is close to normal, and the pulse readings are low. As the applied pressure is increased the blood vessels in the user's body part 108 constrict, increasing the residence time of the blood in the area leading to an increase in the detected pulse intensity. When the applied pressure is increased above the mean pressure exerted during a pulmonary cycle, the blood vessels are further constricted, squeezing blood away from the user's body part 108 and pulse intensity decreases. The pulse intensity against applied pressure constitutes the blood pressure curve and provides important biomarkers such as the diastolic and systolic pressure readings of the user.

The user's blood pressure curve can be calculated from the peak-to-peak pulse intensity of the blood flow signal. The dashed line 414 plots the peaks of the blood flow signal. This shows how the intensity of the blood flow signal varies as a function of the applied pressure. The user's blood pressure, which is typically denoted by the systolic and diastolic measurements, can be extracted from the curve using pre-defined thresholds. In some cases, the blood pressure values can be obtained from the entire blood pressure curve by the means of an analytical function.

FIG. 5 shows an example method of measuring a user's blood pressure. The method can be implemented using an apparatus as shown in any of FIGS. 1 to 3 or by any other suitable apparatus 100.

At block 500 the method comprises starting a blood pressure measurement. The blood pressure measurement can be started through the use of an application or other suitable program. For instance, a user can open a blood pressure monitoring app, or a health care app, or other suitable application. The application or program could be on the apparatus 100 or within the user device that the apparatus 100 is connected, attached or comprised within.

At block 502 the user presses the deformable surface 106 with their body part. For example, the user can press a fingertip against the deformable surface 106. Other body parts 108 could be used in other examples. For instance, the apparatus 100 could be configured to be attached to a user's wrist and a user can change the pressure applied to the deformable surface 106 by pressing the apparatus 100 against their wrist.

In some examples the apparatus 100, or the user device that the apparatus 100 is connected to, attached to or comprised within, can give an indication to a user to start to press against the deformable surface 106. The instructions can comprise information such as how hard to press, the duration for which the user is to maintains the press, any variations in the force of the press and/or any other information.

At block 504 information relating to the applied pressure measurements and/or the user's blood flow measurements can be presented. This can be displayed on the display of the apparatus 100 or of the user device that the apparatus 100 is connected to, attached to or comprised within. The measurement information can be presented in real time or close to real time.

At block 506 it is determined if the full pressure range is done. That is, it is determined if the blood flow measurements have been obtained for the full pressure range. The full pressure range can comprise applied pressures that enable systolic, diastolic and mean blood pressures to be measured.

If, at block 506, it is determined that the full pressure range has not been completed the method proceeds to block 508. At block 508 instructions relating to the pressure applied by the user's body part 108 can be provided. The instructions can indicate to a user if they need to change or maintain the applied pressure. For instance, the instructions can indicate that the applied pressure is to be increased or decreased or if the applied pressure is to be maintained. In some examples the instructions can indicate the time period for which the applied pressure is to be maintained.

The instructions can be provided using any suitable means. The instructions can be provided visually and/or audibly.

Once the instructions have been provided to the user the method returns to block 502 and the user presses the deformable surface 106. The user can press the deformable surface 106 in accordance with the instructions received at block 508. Blocks 502 to 508 can be repeated as many times as needed.

If at block 506 it is determined that the full pressure range has been completed then the method processed to block 510. At block 510 the blood pressure is calculated. The blood pressure is calculated based on the applied pressure and corresponding blood flow signal intensity. The blood pressure measurements can comprise systolic, diastolic and mean pressures or any other values or combinations of values.

An indication of the blood pressure can be presented to the user. In some examples the blood pressure measurement can be stored and/or sent to another device. This can enable logging or recording of the user's blood pressure.

In some examples other measurements could also be made and presented to the user and/or stored with the blood pressure measurements. The other measurements could comprise other biological parameters such as heart rate, blood oxygen levels, or any other suitable parameters.

FIG. 6 shows an example apparatus 100 that is arranged to be attached to a user electronic device 600. The apparatus 100 can be arranged to be removably attached to the user electronic device 600. For example, the apparatus 100 could comprise fastening means that enables the apparatus 100 to be temporarily attached to the user electronic device 600. The fastening means could comprise a clip or any other suitable means. The fastening means can enable the apparatus 100 to be attached to the correct part of the user electronic device 600. For example, the fastening means can enable the apparatus 100 to be attached to the user electronic device 600 so that a light source or the user electronic device 600 can be used as a light source for the apparatus 100.

The user electronic device 600 can be any suitable device. The user electronic device 600 could be a smart phone, a tablet device or any other suitable device.

In the example of FIG. 6 the user electronic device 600 comprises a light source 200 and a light detector 204. The light source 200 and light detector 204 could be part of a camera module or any other suitable part of the user electronic device. The light source 200 and light detector 204 could be arranged for multiple uses. That is, they can be used by the apparatus 100 for blood pressure measurements and can be used by a camera or other module for other purposes.

In the example of FIG. 6 the light source 200 of the user electronic device 600 can provide a broadband beam of light 300. For example, the light source 200 can comprise white LEDs (light emitting diodes) or any other suitable light sources.

The user electronic device 600 can comprise additional components that are not shown in FIG. 6. For example. The user electronic device 600 could comprise a controller. The controller could be configured to received outputs from the light detector 204. The controller can be configured to process the outputs of the light detector 204 to determine the user's blood pressure. In some examples the user electronic device 600 could comprise an output means such as a display and/or a loudspeaker. The output means could be used to provide instructions to the user and/or to provide an indication of the measurements to the user.

In the example of FIG. 6 the apparatus 100 comprises a dichroic filter 302. The broadband beam of light 300 is incident on the dichroic filter 302. The dichroic filter 302 is configured to split the broadband beam of light 300 into a first wavelength beam 304 and a second wavelength beam 306. In the example of FIG. 6 the dichroic filter 302 enables the second wavelength beam 306 to pass through but does not enable the first wavelength beam 304 to pass through. Other types of filters or beam splitters could be used in other examples.

The first wavelength beam 304 is used by the interferometric means 102 to measure displacement of the deformable surface 106. In the example of FIG. 6 the interferometric means 102 comprises a beam splitter 308, a fixed reflective surface 312 and the deformable surface 106.

The interferometric means 102 is arranged so that the first wavelength beam 304 is incident on the beam splitter 308. The beam splitter 308 can comprise any optical components that can be configured to split the first wavelength beam 304 into two parts. A first part is directed towards the fixed reflective surface 312 and the second part is directed towards the deformable surface 106.

The light detector 204 detects the light reflected from the respective arms of the interferometric means 102. This detected light can be used to determine a displacement of the deformable surface 106 and from that determine a pressure applied by the user's body part 108.

The second wavelength beam 306 is used by the blood flow monitoring means 104 to measure blood flow in the user's body part 108. The blood flow monitoring means 104 are configured to enable the second wavelength beam 306 to pass through the deformable surface 106 and onto the user's body part 108. The second wavelength beam 306 can comprise wavelengths that can be used for measuring blood flow. They can comprise red, green and/or infra-red wavelengths. The amount of light of the second wavelength beam 306 that is reflected gives an indication of blood flow in the user's body part 108.

The blood flow monitoring means 104 are configured so that the reflected parts of the second wavelength beam 306 are incident on the light detector 204. This enables the blood flow to be monitored.

FIG. 7 shows another example apparatus 100. In this example the apparatus 100 is an independent device. The independent device can perform the blood pressure measurements without being connected to or attached to another device. The apparatus 100 can comprise all the components that are needed to obtain the measurements of the user's blood pressure. In this example the apparatus 100 comprises the interferometric means 102 and the blood flow monitoring means 104. The apparatus 100 also comprises a light source 200 and a light detector 204.

In some examples the apparatus 100 can comprise a controller or any other suitable processing means. The controller can be configured to process the output signals from the light detector 204 so as to determine a user's blood pressure. In other examples the apparatus 100 can be configured to transmit the signals to another device to enable the processing of the signals. For instance, the apparatus 100 could be connected to a processing device via a wireless communication link.

In the example of FIG. 7 the user apparatus 100 comprises a light source 200 and a light detector 204. The light source 200 can provide light at any suitable wavelengths. In this example the light source 200 only needs to provide light for the apparatus 100 so the light might only be used for the purpose of measuring blood pressure. The light source 200 can therefore be selected to provide wavelengths of light that are well suited for the purpose of the making the blood flow measurements and the interferometric measurements.

In the example of FIG. 7 the light sources 200 comprise a green LED 700 and an infra-red LED 702. These can provide a beam of green light 704 and a beam of infra-red light respectively 706. These wavelengths of light might be used because they are well suited for the blood flow monitoring. Other wavelengths of light could be used in other examples. In such examples there could be other types or combinations of LEDs. For example, a red LED could be used with a green LED and an infra-red LED.

In the example of FIG. 7 the apparatus 100 comprises optical components 708. The optical components comprise a combination of dichroic mirrors and notch filters. The notch filters are configured to allow a narrow band of frequencies to be selected for use by the interferometric means 102.

In this case the optical components 708 are arranged so that some of the green light 704 is used by the interferometric means 102 and the infra-red light and some of the green light 704 is used by the blood flow monitoring means 104.

The green light 704 is used by the interferometric means 102 to measure displacement of the deformable surface 106. In the example of FIG. 7 the interferometric means 102 comprises a beam splitter 308, a fixed reflective surface 312 and the deformable surface 106.

The green light 704 from the notch filter 708 is incident on the beam splitter 308. The beam splitter 308 can comprise any optical components that can be configured to split the green light 704 into two parts. A first part is directed towards the fixed reflective surface 312 and the second part is directed towards the deformable surface 106.

The light detector 204 detects the light reflected from the respective arms of the interferometric means 102. In these examples the light detector 204 comprises a pair of photodiodes 710. The photodiodes 710 can be configured to detect green light and infra-red light.

The infra-red light 706 and the green light 704 is used by the blood flow monitoring means 104 to measure blood flow in the user's body part 108. The blood flow monitoring means 104 are configured to enable the infra-red light 706 and the green light 704 to pass through the deformable surface 106 and onto the user's body part 108. The infra-red light 706 and the green light 704 that is reflected from the user's body part 108 is detected by the photodiodes 710.

The photodiodes 710 are configured to detect both the light from the interferometric means 102 and the light from the blood flow monitoring means 104. The same wavelength of light can be used by both the interferometric means 102 and the blood flow monitoring means 104. In this case the output of the photodiodes 710 will comprise a high frequency component and a low frequency component. The high frequency component can correspond to the output of the interferometric means 102 and the low frequency component can correspond to the output of the blood flow monitoring means 104. The respective components can be analyzed individually to provide a blood pressure measurement.

FIG. 8 shows an example method that can be used in some examples of the disclosure. The method can be implemented by an apparatus 100 as shown or in any of FIGS. 1 to 4 and 6 to 7. In some examples the method could be implemented by a user electronic device that comprises an apparatus 100 or that is connected, attached or comprised within the apparatus 100. In some examples the method can be performed by a device that is configured to receive signals from another apparatus 100. For example, a processing device could receive signals from an apparatus using a wireless communication link or any other suitable means. The method can be performed by a controller. An example of a controller is shown in FIG. 10.

At block 800 the method comprises obtaining an interferometric signal. The interferometric signal can be obtained from interferometric means 102 as shown in FIG. 1 or from any other suitable source. The interferometric signal indicates a deformation of deformable surface 106 where the deformation is caused by a user's body part pressing against the deformable surface 106.

The interferometric signal can comprise an output from a light detector 204. The interferometric signal can comprise an electric signal that is provided by a light detector 204 or any other suitable means.

At block 802 the method comprises obtaining a blood flow monitoring signal where the signal is obtained from one or more sensors configured to monitor blood flow in the user's body part 108. The sensors can comprise photoplethysmography sensors or any other suitable type of sensors.

The blood flow monitoring signal can comprise an output from a light detector 204. The blood flow monitoring signal can comprise an electric signal that is provided by a light detector 204 or any other suitable means.

At block 804 the method can comprise processing the interferometric signal and the blood flow monitoring signal to calculate the user's blood pressure.

The interferometric signal and the blood flow monitoring signal are obtained from measurements made within simultaneous time windows. That is, the interferometric signal is obtained over a time window so as to enable a user to apply different levels of pressure to the deformable surface 106. The blood flow monitoring signal is obtained at while the user is pressing against the deformable surface 106 so that the blood flow can be correlated with the pressure applied at the time the blood flow measurement was taken.

The blood flow monitoring signal is obtained for a range of deformations of the deformable surface 106. The range of deformations can be obtained by the user pressing the deformable surface 106 with different levels of pressure.

The range of deformations and applied pressures used to cause the deformation of the deformable surface 106 correspond to a range of measurable blood pressures. The range of measurable blood pressures can comprise diastolic pressure, mean pressure, systolic pressure, and/or any other relevant pressure levels.

In some examples the user can be provided with instructions to enable the blood pressure measurements to be made. For example, the apparatus 100, or any other suitable device, can be configured to provide instructions to the user. The instructions can indicate a force to be applied to the deformable surface 106 by the user's body part 108 such that the range of deformations are achieved and/or can indicate any other suitable information.

FIGS. 9A to 9C show example systems 900 that can be used to implement examples of the disclosure. Each of the systems 900 comprise an apparatus 100. The apparatus 100 can be as shown in any of FIGS. 1 to 4 or 6 and 7. The apparatus 100 comprises interferometric means 102 and blood flow monitoring means 104. The systems 900 also comprise means for processing an output of the interferometric means 102 and an output of the blood flow monitoring means 104 to calculate the user's blood pressure. In the examples of FIGS. 9A to 9C the means for processing comprises a controller 902. Other means could be used in other examples.

In the example of FIG. 9A the apparatus 100 is an independent apparatus 100. The apparatus 100 can perform the measurements of the applied pressure and the user's blood flow and can also process the output signals so as to calculate the user's blood pressure. The independent apparatus 100 can calculate the user's blood pressure without using any other device.

In this example the apparatus 100 comprises a light detector 204 and a controller 902. The apparatus 100 could also comprise other components that are not shown in FIG. 9A. The light detector 204 is configured to receive optical signals from the interferometric means 102 and the blood flow monitoring means 104. The controller 902 is configured to receive the outputs from the light detector 204. The controller 902 is configured to process the outputs from the light detector 204 to calculate the user's blood pressure.

In the example system 900 of FIG. 9B the apparatus 100 is configured to perform the measurements of the applied pressure and the user's blood flow. The apparatus 100 can then provide output signals to a user electronic device 600 to enable the user electronic device 600 to process the output signals so as to calculate the user's blood pressure.

In this example the apparatus 100 comprises a light detector 204 and a transmitter 904. The apparatus 100 could also comprise other components that are not shown in FIG. 9B. The light detector 204 is configured to receive optical signals from the interferometric means 102 and the blood flow monitoring means 104. The outputs from the light detector 204 are provided to the transmitter 904. The signals are encoded or otherwise processed for transmission to the user electronic device 600.

The signals can be transmitted to the user electronic device 600 using any suitable communication protocol.

The user electronic device 600 comprises a receiver 906 and a controller 902. The user electronic device 600 could also comprise other components that are not shown in FIG. 9B. The receiver 906 is configured to receive the signals from the apparatus 100. The signals can be decoded or otherwise processed. The received signals are provided to the controller 902. The controller 902 is configured to process the received signals to calculate the user's blood pressure.

In the example system 900 of FIG. 9C the apparatus 100 is configured to be attached to a user electronic device 600 or to be comprised within a user electronic device 600. In this example the apparatus 100 does not comprise a light detector 204. In this example the apparatus 100 comprises interferometric means 102 and blood flow monitoring means 104. The outputs of the interferometric means 102 and blood flow monitoring means 104 are provided as optical signals.

The apparatus 100 is configured relative to the user electronic device 600 so that the optical signals from the interferometric means 102 and the blood flow monitoring means 104 are provided as an input to the light detector 204 within the user electronic device 600.

In this example user electronic device 600 comprises a light detector 204 and controller 902. The user electronic device 600 could also comprise other components that are not shown in FIG. 9C. The controller 902 is configured to receive the outputs from the light detector 204. The controller 902 is configured to process the outputs from the light detector 204 to calculate the user's blood pressure.

FIGS. 9A to 9C shows a selection of arrangements that could be used for systems in examples of the disclosure. Other arrangements of an apparatus 100 and processing means such as a controller 902 could be used in other examples.

Examples of the disclosure therefore provide an apparatus 100 that can be used by a consumer to measure their own blood pressure. The apparatus 100 comprises no moving parts so provides for a durable and reliable device.

The apparatus 100 is easy to use as a user simply needs to press their finger or other suitable body part against the deformable surface 106. This might be useful for people with reduced strength or dexterity. Instructions to the user can also be provided to assist the user. Such instructions will be simple and straightforward for a user to understand as it would only involve how hard the user presses their finger.

The apparatus 100 does not need to be calibrated by a user before use (the apparatus could be calibrated after manufacturing, for example, before the apparatus is shipped). This is particularly beneficial for a consumer device where calibrations might be too complicated for some user or might make the measurements more inconvenient for a user to perform.

FIG. 10 shows an example controller 902. The controller 902 can be suitable for use in apparatus 100 and systems 900 as described herein.

Implementation of the controller 902 may be as controller circuitry. The controller 902 may be implemented in hardware alone, have certain aspects in software including firmware alone or can be a combination of hardware and software (including firmware).

As illustrated in FIG. 10 the controller 902 can be implemented using instructions that enable hardware functionality, for example, by using executable instructions 1006 in a general-purpose or special-purpose processor 1002 that may be stored on a machine readable storage medium (disk, memory etc.) to be executed by such a processor 1002.

The processor 1002 is configured to read from and write to the memory 1004. The processor 1002 may also comprise an output interface via which data and/or commands are output by the processor 1002 and an input interface via which data and/or commands are input to the processor 1002.

The memory 1004 stores instructions 1006, program or code that controls the operation of the apparatus 100 when loaded into the processor 1002. The instructions 1006, program or code, provide the logic and routines that enable the apparatus 100 to perform the methods illustrated in the accompanying FIGS. The processor 1002 by reading the memory 1004 is able to load and execute the instructions 1006, program or code.

The apparatus 100 comprises:
at least one processor 1002; and
   at least one memory 1004 storing instructions 1006 that, when executed by the at least one processor 1002, cause the apparatus 100 at least to:
   obtaining 800 an interferometric signal where the interferometric signal indicates a deformation of deformable surface 106 where the deformation is caused by a user's body part 108 pressing against the deformable surface 106;
   obtaining 802 a blood flow monitoring signal where the signal is obtained from one or more sensors configured to monitor blood flow in the user's body part; and
   processing 804 the interferometric signal and the blood flow monitoring signal to calculate the user's blood pressure.

In some examples there is a system comprising:
an apparatus as described herein;
   and
means for processing an output of the interferometric means and an output of the blood flow monitoring means to calculate the user's blood pressure.

As illustrated in FIG. 10 the instructions 1006, program or code can arrive at the apparatus 100 via any suitable delivery mechanism 1008. The delivery mechanism 1008 can be, for example, a machine readable medium, a computer-readable medium, a non-transitory computer-readable storage medium, a computer program product, a memory device, a record medium such as a Compact Disc Read-Only Memory (CD-ROM) or a Digital Versatile Disc (DVD) or a solid-state memory, an article of manufacture that comprises or tangibly embodies the computer program, the instructions 1006, program or code. The delivery mechanism may be a signal configured to reliably transfer the computer program the instructions 1006, program or code. The apparatus 100 may propagate or transmit the computer program the instructions 1006, program or code as a computer data signal.

The term “non-transitory,” as used herein, is a limitation of the medium itself (that is, tangible, not a signal) as opposed to a limitation on data storage persistency (that is, RAM vs. ROM).

Computer program instructions for causing an apparatus to perform at least the following or for performing at least the following:
obtaining 800 an interferometric signal where the interferometric signal indicates a deformation of deformable surface 106 where the deformation is caused by a user's body part 108 pressing against the deformable surface 106;
obtaining 802 a blood flow monitoring signal where the signal is obtained from one or more sensors configured to monitor blood flow in the user's body part; and
processing 804 the interferometric signal and the blood flow monitoring signal to calculate the user's blood pressure.

The computer program instructions may be comprised in a computer program, a non-transitory computer readable medium, a computer program product, a machine readable medium. In some but not necessarily all examples, the computer program instructions may be distributed over more than one computer program.

Although the memory 1004 is illustrated as a single component/circuitry it may be implemented as one or more separate components/circuitry some or all of which may be integrated/removable and/or may provide permanent/semi-permanent/dynamic/cached storage.

Although the processor 1002 is illustrated as a single component/circuitry it may be implemented as one or more separate components/circuitry some or all of which may be integrated/removable. The processor 1002 may be a single core or multi-core processor.

References to ‘computer-readable storage medium’, ‘computer program product’, ‘tangibly embodied computer program’ etc. or a ‘controller’, ‘computer’, ‘processor’ etc. should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other processing circuitry. References to computer program, instructions, code etc. should be understood to encompass software for a programmable processor or firmware such as, for example, the programmable content of a hardware device whether instructions for a processor, or configuration settings for a fixed-function device, gate array or programmable logic device etc.

As used in this application, the term ‘circuitry’ may refer to one or more or all of the following:
(a) hardware-only circuitry implementations (such as implementations in only analog and/or digital circuitry) and
(b) combinations of hardware circuits and software, such as (as applicable):
(i) a combination of analog and/or digital hardware circuit(s) with software/firmware and
(ii) any portions of hardware processor(s) with software (including digital signal processor(s)), software, and memory or memories that work together to cause an apparatus, such as a mobile phone or server, to perform various functions and
(c) hardware circuit(s) and or processor(s), such as a microprocessor(s) or a portion of a microprocessor(s), that requires software (for example, firmware) for operation, but the software may not be present when it is not needed for operation.

This definition of circuitry applies to all uses of this term in this application, including in any claims. As a further example, as used in this application, the term circuitry also covers an implementation of merely a hardware circuit or processor and its (or their) accompanying software and/or firmware. The term circuitry also covers, for example and if applicable to the particular claim element, a baseband integrated circuit for a mobile device or a similar integrated circuit in a server, a cellular network device, or other computing or network device.

The blocks illustrated in the accompanying FIGS. may represent steps in a method and/or sections of code in the computer program or instruction 1006. The illustration of a particular order to the blocks does not necessarily imply that there is a required or preferred order for the blocks and the order and arrangement of the block may be varied. Furthermore, it may be possible for some blocks to be omitted.

Where a structural feature has been described, it may be replaced by means for performing one or more of the functions of the structural feature whether that function or those functions are explicitly or implicitly described.

The apparatus can be provided in an electronic device, for example, a mobile terminal, according to an example of the present disclosure. It should be understood, however, that a mobile terminal is merely illustrative of an electronic device that would benefit from examples of implementations of the present disclosure and, therefore, should not be taken to limit the scope of the present disclosure to the same. While in certain implementation examples, the apparatus can be provided in a mobile terminal, other types of electronic devices, such as, but not limited to: mobile communication devices, hand portable electronic devices, wearable computing devices, portable digital assistants (PDAs), pagers, mobile computers, desktop computers, televisions, gaming devices, laptop computers, cameras, video recorders, GPS devices and other types of electronic systems, can readily employ examples of the present disclosure. Furthermore, devices can readily employ examples of the present disclosure regardless of their intent to provide mobility.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to “comprising only one . . . ” or by using “consisting”.

In this description, the wording ‘connect’, ‘couple’ and ‘communication’ and their derivatives mean operationally connected/coupled/in communication. It should be appreciated that any number or combination of intervening components can exist (including no intervening components), i.e., so as to provide direct or indirect connection/coupling/communication. Any such intervening components can include hardware and/or software components.

As used herein, the term “determine/determining” (and grammatical variants thereof) can include, not least: calculating, computing, processing, deriving, measuring, investigating, identifying, looking up (for example, looking up in a table, a database or another data structure), ascertaining and the like. Also, “determining” can include receiving (for example, receiving information), accessing (for example, accessing data in a memory), obtaining and the like. Also, “determine/determining” can include resolving, selecting, choosing, establishing, and the like.

In this description, reference has been made to various examples. The description of features or functions in relation to an example indicates that those features or functions are present in that example. The use of the term ‘example’ or ‘for example’ or ‘can’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’, ‘can’ or ‘may’ refers to a particular instance in a class of examples. A property of the instance can be a property of only that instance or a property of the class or a property of a sub-class of the class that includes some but not all of the instances in the class. It is therefore implicitly disclosed that a feature described with reference to one example but not with reference to another example, can where possible be used in that other example as part of a working combination but does not necessarily have to be used in that other example.

As used herein, “at least one of the following . . . ” and “at least one of . . . ” and similar wording, where the list of two or more elements are joined by “and” or “or” mean at least any one of the elements, or at least any two or more of the elements, or at least all the elements.

Although examples have been described in the preceding paragraphs with reference to various examples, it should be appreciated that modifications to the examples given can be made without departing from the scope of the claims.

Features described in the preceding description may be used in combinations other than the combinations explicitly described above.

Although functions have been described with reference to certain features, those functions may be performable by other features whether described or not.

Although features have been described with reference to certain examples, those features may also be present in other examples whether described or not.

The term ‘a’, ‘an’ or ‘the’ is used in this document with an inclusive not an exclusive meaning. That is any reference to X comprising a/an/the Y indicates that X may comprise only one Y or may comprise more than one Y unless the context clearly indicates the contrary. If it is intended to use ‘a’, ‘an’ or ‘the’ with an exclusive meaning then it will be made clear in the context. In some circumstances the use of ‘at least one’ or ‘one or more’ may be used to emphasis an inclusive meaning but the absence of these terms should not be taken to infer any exclusive meaning.

The presence of a feature (or combination of features) in a claim is a reference to that feature or (combination of features) itself and also to features that achieve substantially the same technical effect (equivalent features). The equivalent features include, for example, features that are variants and achieve substantially the same result in substantially the same way. The equivalent features include, for example, features that perform substantially the same function, in substantially the same way to achieve substantially the same result.

In this description, reference has been made to various examples using adjectives or adjectival phrases to describe characteristics of the examples. Such a description of a characteristic in relation to an example indicates that the characteristic is present in some examples exactly as described and is present in other examples substantially as described.

The above description describes some examples of the present disclosure however those of ordinary skill in the art will be aware of possible alternative structures and method features which offer equivalent functionality to the specific examples of such structures and features described herein above and which for the sake of brevity and clarity have been omitted from the above description. Nonetheless, the above description should be read as implicitly including reference to such alternative structures and method features which provide equivalent functionality unless such alternative structures or method features are explicitly excluded in the above description of the examples of the present disclosure.

Whilst endeavoring in the foregoing specification to draw attention to those features believed to be of importance it should be understood that the Applicant may seek protection via the claims in respect of any patentable feature or combination of features hereinbefore referred to and/or shown in the drawings whether or not emphasis has been placed thereon.

1-16. (canceled)

17. An apparatus comprising:
an interferometer comprising at least one deformable surface wherein the deformable surface is at least partially reflective and is configured to be deformed by a user's body part and wherein the interferometer is configured to measure deformation of the deformable surface caused by the user's body part;
a blood flow monitor configured to monitor blood flow in the user's body part;
at least one processor; and
at least one memory storing instructions that, when executed by the at least one processor, cause the apparatus at least to:
provide an output of the interferometer and an output of the blood flow monitor, wherein the output of the interferometer and the output of the blood flow monitor are configured to be used to calculate the user's blood pressure.

18. An apparatus as claimed in claim 17 wherein the blood flow monitor comprises one or more photoplethysmography sensors.

19. An apparatus as claimed in claim 18 wherein the one or more photoplethysmography sensors comprise a light source configured to direct light towards the user's body part and one or more sensors configured to detect light scattered from the user's body part.

20. An apparatus as claimed in claim 17 wherein the blood flow monitor is configured to use beams of light within a wavelength range, and wherein at least part of the at least one deformable surface is at least partially transparent to the beams of light within the wavelength range.

21. An apparatus as claimed in claim 17 comprising one or more light sources wherein the one or more light sources are configured to provide beams of light for at least one of:
the interferometer; or
the blood flow monitor.

22. An apparatus as claimed in claim 21 wherein the one or more light sources are configured to provide beams of light in a first wavelength range for the interferometer and beams of light in a second wavelength range, different to the first wavelength range, for the blood flow monitor.

23. An apparatus as claimed in claim 17 wherein the interferometer comprises a sensor configured to detect light reflected from the deformable surface and light reflected from a fixed reflective surface.

24. An apparatus as claimed in claim 17 wherein the apparatus is further caused to provide instructions to the user to indicate a force to be applied to the deformable surface by the user's body part.

25. An apparatus as claimed in claim 17 wherein the apparatus is at least one of:
comprised within a user electronic device;
an attachment for a user electronic device; or
an independent device.

26. An apparatus comprising:
at least one processor; and
at least one memory storing instructions that, when executed by the at least one processor, cause the apparatus at least to:
obtain an interferometric signal where the interferometric signal indicates a deformation of a deformable surface where the deformation is caused by a user's body part pressing against the deformable surface;
obtain a blood flow monitoring signal where the blood flow monitoring signal is obtained from one or more sensors configured to monitor blood flow in the user's body part; and
process the interferometric signal and the blood flow monitoring signal to calculate the user's blood pressure.

27. An apparatus as claimed in claim 26 wherein the interferometric signal and the blood flow monitoring signal are obtained from measurements made within simultaneous time windows.

28. An apparatus as claimed in claim 26 wherein the blood flow monitoring signal is obtained for a range of deformations of the deformable surface.

29. An apparatus as claimed in claim 28 wherein the range of deformations of the deformable surface correspond to a range of pressures comprising at least one of:
diastolic pressure;
mean pressure; or
systolic pressure.

30. An apparatus as claimed in claim 28 wherein the apparatus is further caused to provide instructions to the user to indicate a force to be applied to the deformable surface by the user's body part such that the range of deformations are achieved.

31. An apparatus as claimed in claim 17 wherein the at least one memory and the instructions stored therein are configured to, with the at least one processor, further cause the apparatus to:
process the output of the interferometer and the output of the blood flow monitor; and
calculate the user's blood pressure using the output of the interferometer and the output of the blood flow monitor.

32. A method comprising:
obtaining an interferometric signal where the interferometric signal indicates a deformation of a deformable surface where the deformation is caused by a user's body part pressing against the deformable surface;
obtaining a blood flow monitoring signal where the blood flow monitoring signal is obtained from one or more sensors configured to monitor blood flow in the user's body part; and
processing the interferometric signal and the blood flow monitoring signal to calculate the user's blood pressure.

33. A method as claimed in claim 32 wherein the interferometric signal and the blood flow monitoring signal are obtained from measurements made within simultaneous time windows.

34. A method as claimed in claim 32 wherein the blood flow monitoring signal is obtained for a range of deformations of the deformable surface.

35. A method as claimed in claim 34 wherein the range of deformations of the deformable surface correspond to a range of pressures comprising at least one of:
diastolic pressure;
mean pressure; or
systolic pressure.

36. A method as claimed in claim 34 further comprising:
providing instructions to the user to indicate a force to be applied to the deformable surface by the user's body part such that the range of deformations are achieved.
